(19) United States
(12) Patent Application Publication
(10) Pub. No.: US 2020/0101326 A1
(43) Pub. Date: Apr. 2, 2020

(54) MONOLITHINC PHOTODETECTOR FOR DOSIMETER

(71) Applicant: AngioDynamics, Inc., Latham, NY (US)

(72) Inventor: Clint Zeringue, Houston, TX (US)

(73) Assignee: AngioDynamics, Inc., Latham, NY (US)

(21) Appl. No.: 16/589,210

(22) Filed: Oct. 1, 2019

Related U.S. Application Data
(62) Division of application No. 15/135,576, filed on Apr. 22, 2016.
(60) Provisional application No. 62/150,852, filed on Apr. 22, 2015.

(51) Int. Cl. A61N 5/10; G01T 1/02

(52) U.S. Cl. CPC A61N 5/1071; A61N 5/1067; A61N 2005/1003; G01T 1/023; G01T 1/026

(57) ABSTRACT

Detectors, system and methods for measuring radiation using plastic scintillating sensors with a detector unit that uses photodiodes and MPPC detectors instead of a CCD camera, and thus avoids the use of all free space optics.

### PRIOR RELATED APPLICATIONS

This application claims priority to 62/150,852, entitled MONOLITHIC PHOTODIODE DETECTOR FOR DOSIMETER, filed Apr. 22, 2015, incorporated by reference herein in its entirety for all purposes.

### FEDERALLY SPONSORED RESEARCH STATEMENT

Not applicable.

### FIELD OF THE DISCLOSURE

The disclosure generally relates to photodetectors for use in radiation measurements.

### BACKGROUND OF THE DISCLOSURE

A scintillator is a special material that exhibits scintillation—the property of luminescence when excited by ionizing radiation. Luminescent materials, when struck by an incoming particle, absorb its energy and scintillate, in other words they reemit the absorbed energy in the form of light.

A scintillation detector or scintillation counter is obtained when a scintillator is coupled to a light sensor such as a photomultiplier tube (PMT), photodiode, including arrays of photodiodes, operating in Geiger mode also known as an Avalanche Photodiode (APD), PIN diode, Multi-Pixel Photon Detector (MPPC), or charged-coupled device (CCD)-based photodetector. The light sensor will absorb the light emitted by the scintillator and generate electrons via the photoelectric effect. The subsequent multiplication of those electrons (sometimes called photo-electrons) results in an electrical pulse that can be analyzed and provides meaningful information about the particle that originally struck the scintillator. In this way, the original amount of absorbed energy can be detected or counted.

The term “plastic scintillator” typically refers to a scintillating material where the primary fluorescent emitter, called a fluor, is suspended in a solid polymer matrix. While this combination is typically accomplished through the dissolution of the fluor prior to bulk polymerization, the fluor is sometimes associated with the polymer directly, either covalently or through coordination, as is the case with many Li₆ plastic scintillators. Polyethylene naphthalate has been found to scintillate without any additives and is expected to replace existing plastic scintillators due to its higher performance and lower price.

The advantages of plastic scintillators include fairly high light output and a relatively quick signal, with a decay time between 2-4 nanoseconds. The biggest advantage of plastic scintillators, though, is their ability to be shaped, through the use of molds or other means, into almost any desired form with a high degree of durability.

In the field of medical radiation therapy, plastic scintillation detectors are used to convert radiation energy into light energy, and the light photons are counted to accurately determine the radiation dose. The scintillating plastic must transfer its photons to a device that can read them, which is commonly done by coupling one or more scintillating fibers to one or more plastic optical fibers (POF). The POF is then connected to a device that can read and analyze the optical output.

RadiaDyne, for example, has designed a radiation detection system that uses a CCD camera to measure the light produced by a plastic scintillating fiber that is small enough to be inserted in the human body. The cable is attached to a balloon that can be inserted into a body orifice, expanded and used to compress tissue so that it remains immobile for e.g., external beam radiation therapy. This is particularly useful for prostate irradiation, as the prostate otherwise is subject to considerable movement, necessitating an increase in treatment margins and the possibility of significant irradiation of otherwise healthy tissue surrounding the prostate.

A CCD is a photoelectric semiconductor with compact size, light-weight, and high spatial resolution. Even though CCDs have various advantages compared to other light detectors, they were originally not favored by some because of their poor signal to noise ratio (SNR) at low count rates. This problem was mainly caused by dark current, which is primarily affected by the CCD surface temperature. All CCDs benefit from working at lower temperatures, because thermal energy alone is enough to excite extraneous electrons into the image pixels and these cannot be distinguished from the actual image photoelectrons. This process generates noise and is called ‘dark current.’

Recently, a Peltier-based cooling system was developed, and a CCD camera was equipped with this cooling system. Thus, the temperature of the detector was lower and the dark current was significantly reduced, and many are now developing dosimeters based on the plastic scintillating fiber and the cooled CCD detector. Indeed, Louis Amchambault from Laval University (QUEBEC CA) has concluded that the CCD provides the “optimal photodetector,” possessing sufficient sensitivity, allowing the simultaneous measurement of more than 3,000 dose signals, and having built-in color separation and sufficiently stability.

However, the utility of scientific cameras is limited by their sensitivity to stray radiation within the linear accelerator bunker. This manifests as distorted pixel intensity values, or ‘spikes’, in the recorded images, and typically necessitates that the detector unit be placed in a separate location, shielded from as much stray radiation as possible. This, then necessitates the provision of a substantial cable to couple the detector to the dosimeter, which is often quite some distance away, and this cable introduces another source of noise, transmission loss, and possible breakdown and makes installation quite complex and in some instances impossible.

Further, calibration of the device is complex, and typically is done at manufacture, using a calibrated light source, optical alignment techniques, assembly of free-space optical components that guide light onto the CCD camera, and specialized software drivers for interfacing with the CCD camera. The specialized software drivers introduce a degree of dependency on specific CCD manufacturers and/or instrument driver software. Thus, replacing the CCD camera with a different model or manufacturer will also involve replacing the instrument drivers and modification of software.

Additionally, given that each component in the CCD system carries a degree of variability in its response to light, the overall uncertainty associated with calibration of the device increases.

Also, the CCD device is large, complex and not particularly robust, subject to damage during shipping and installation as the free-space optical components that guide light onto the CCD can move, thus causing misalignment. The free-space optical components are required to guide light from the exit of the POF onto the detector element of the CCD. The “free-space optical components” typically consist of a series of mirrors, color filters, and lenses whereby light is split into two distinct color regions and focused on the CCD image plane. The measurement process requires that the light exiting the POF be split into two color regions. Given the high numerical aperture (NA) typically associated with POF, the light exiting the POF spreads dramatically over even a few inches. As a result, the light passing through the optical train is clipped by the various free-space optical components. Therefore, passage through the free-space optical components results in a significant loss of the signal light entering the system. Further, due to the low-level of light produced by the scintillator, any additional light loss in the detection process decreases the sensitivity of the device and limits dosimetric accuracy.

In an effort to minimize this effect, expensive CCD cameras that exhibit very low pixel noise, uniformity of response, and high quantum efficiency are required. In addition, the image acquisition time on the CCD is increased to about 20 seconds to allow sufficient build-up of light intensity and adequate signal-to-noise ratio (SNR). Thus, the CCD-based system is not very fast, and these delays detract from real-time dosimetry.

One major issue we discovered on prototyping a CCD based system was that due to user error it is possible that too much light can enter the system and damage the CCD internally. With photodiodes this is not an issue because they do not get damaged when exposed to ambient light conditions.

Finally, the CCD based cameras are large, contributing to the overall footprint of the system, as well as manufacturing costs.

Therefore, a need exists for better methods, devices and systems for detecting and measuring the signal from plastic scintillating fibers. The ideal device would have one or more of the following qualities: be smaller, easily calibrated, not be temperature sensitive, be more robust or impervious to shipping and installation misalignments, have a greater signal to noise ratio, be able to be used with any computer, be more economically feasible, and be amenable to use on site.

### SUMMARY OF THE DISCLOSURE

Generally speaking, the invention is directed to dosimeters that use monolithic photodiode detectors instead of the larger, less robust, difficult to calibrate and temperature sensitive CDD cameras.

The plastic scintillator detector or “PSD” typically has only a small segment of plastic scintillator material, which is connected to fiber optic cable for carrying the signal to the photodetector. Preferably this connection is a direct abutment, without adhesives, as taught e.g., in US20120281945. Also preferred, the cable may be inserted into a balloon specially designed for radiation oncology use, such as e.g., the prostate cancer balloon described in U.S. Pat. No. 7,976,497, et seq. and the balloons of U.S. Pat. Nos. 9,227,084, 8,500,618, 9,126,035. Each of these patents is incorporated by reference herein in its entirety for all purposes herein.

The connecting end of the fiber optic cable is fed into a robust fiber alignment connector, preferably a SubMiniature version A or “SMA” connector. This is then connected to a photodiode, preferably a RGB photodiode, which is affixed to a printed circuit board or “PCB” in an SMA receptacle or mount, which lockingly receives the SMA connector, typically with a threaded connector.

The photodiode can also be coupled to the fiber optic cable by directly mounting the fiber onto the surface of the detector (via e.g. epoxy, bulkhead connector and the like) with virtually zero light loss. Alternatively, a fiber optic splitter, which splits the light input equally into two other fibers, can be attached to the fiber optic cable. Each of these “daughter” fibers can then be mounted onto a single photodiode with a color filter adhered to the detector surface. Alternatively, a dichroic prism or a crossed dichroic prism may be used, to split the light input into two-color or three-color regions respectively. In this case, the fiber optic cable is attached to one side of the dichroic prism surface (via e.g. epoxy, bulkhead connector, cylindrical housing, and the like). In the case of the two-color dichroic prism detection system, two photodiodes or two MPPC detectors are then attached to the reflection and transmission sides of the dichroic prism (via. e.g. epoxy, bulkhead connector and the like). Likewise, in the crossed-dichroic, three-color detection system, three photodiodes or three MPPC detectors are then attached to the reflection and transmission sides (via. e.g. epoxy, bulkhead connector, and the like). These approaches have the added advantage of selecting low-noise, high quantum efficiency photodiodes or MPPC detectors yielding greater sensitivity and customizable color filters.

These approaches result in a dramatic increase in signal to noise ratio because the light exiting the POF never passes through any free-space optical train and the entire path is monolithic making it less sensitive to shifting during shipment, vibration, or transportation of the device. Thermal effects are also easily de-coupled using specialized electronic circuit design.

The RGB photodiode is preferred because it allows one to measure up to three-color channels on a single chip. Preferably, the RGB photodiode has three closely packed silicon detectors, each with a filter for allowing a given spectrum of light onto the respective photodiode. However, other photodiodes could be used, if desired.

For example, Silicon photomultipliers, often called “SiPM” in the literature, are silicon single photon sensitive devices built from an avalanche photodiode (APD) array on common Si substrate. The idea behind this device is the detection of single photon events in sequentially connected Si APDs.

As another example, the MPPC (multi-pixel photon counter) is a type of SiPM. It is a photon-counting device using multiple APD (avalanche photodiode) pixels operating in Geiger mode. Although the MPPC is essentially an opto-semiconductor device, it has excellent photon-counting capability and can be used in various applications for detecting extremely weak light at the photon counting level. The MPPC operates on low voltage and features high gain, high photon detection efficiency, high-speed response, excellent time resolution, and wide spectral response range. It achieves the performance that is required in photon-counting at a high level. The MPPC is also immune to magnetic fields, highly resistant to mechanical shocks and the like, which are advantages unique to solid-state devices.

A PCB with the various components attached thereto is contained inside the housing of a clinical detector unit, with an additional layer of shielding around the PCB. This small unit contains all of the software, firmware and hardware needed to read the light signals arriving via the fiber optic cable, convert them to digital signals, and then sends that data via wire or wirelessly to any suitable computer for calculation of dosages and display and/or recording. The unit preferably also has its own display, however, and dosage and other system information can be displayed there as well. Wireless communication is greatly preferred because of the difficulty and bulk of using cables.

No temperature calibration is needed with this new device because it is not temperature sensitive, unlike the prior art CCD based systems. Although the photodiodes and MPPC detectors do exhibit some thermal sensitivity, specialized circuitry and sensors are embedded within the PCB, to account for any variation in thermal changes inside the detector. Further, the photodiodes and MPPC detectors are less noisy, more compact allowing for more efficient shielding, and thus can be housed in the same room as the patient, eliminating the needed for a delivery cable. In addition, real-time dosimetry is achieved using photodiodes and MPPC detectors as the dose is measured in sub-second and/or second intervals.

The invention includes and one or more of the following embodiments, in any combination(s) thereof:

A dosimetry detector system, comprising:

a radiation sensor cable having an opaque jacket encasing a plastic scintillation fiber abutting a fiber optic cable, and terminating in a fiber alignment ferrule;

a detector unit having an opaque and shielded housing containing a printed circuit board on which is secured a photodiode, preferably an RGB photodiode or MPPC, inside a connector that reversibly couples to said fiber alignment ferrule such that an end of said fiber optic cable directly abuts said photodiode;

said detector unit having a communication system for communicating data from said photodiode to a separate computer;

said detector unit not requiring temperature correction. Preferably, the unit doesn't require a cable for connecting to said computer either, and is capable of real time (<1 second) response times.

A dosimeter detector, comprising:

a detector unit having an opaque and shielded housing containing a printed circuit board on which is secured an RBG photodiode inside a connector that reversibly couples to a fiber alignment ferrule such that an end of a separate fiber optic cable of a dosimeter sensor directly abuts said RBG photodiode;

said detector unit having a wireless communication system for communicating data from said RBG photodiode to a separate computer;

said detector unit not requiring temperature correction or a cable for connecting to said computer;

wherein said detector unit can measure a radiation dosage via said dosimeter sensor in less than 1 second.

A method of patient treatment with radiation, said method comprising:

inserting the radiation sensor cable as herein described into a patient, such that said plastic scintillating fiber is at or near a tumor;

coupling said connector to said fiber alignment ferrule;

irradiating said tumor;

measuring a dosage of said irradiation using said detector unit; and

ceasing said irradiation when said measured dosage reaches a desired level of radiation.

A method of patient treatment with radiation, said method comprising:

inserting a radiation sensor cable and rectal balloon into a rectum of a patient, such that said plastic scintillating fiber is at or near a cancerous prostate;

inflating said balloon so as to compress and immobilize said prostate;

coupling said connector to said fiber alignment ferrule;

irradiating said prostate;

measuring a dosage of said irradiation using said detector unit; and

ceasing said irradiation when said measured dosage reaches a desired level of radiation.

In those preferred embodiments with a barcode reader, the radiation sensor cable or dosimeter would have a barcode, and the method would also include the step of reading the barcode into the detector unit, thus providing calibration information to the system, and possibly also patient and/or treatment information.

A detector unit, system or method as herein described wherein said plastic scintillation fiber directly abuts said fiber optic cable without glue therebetween.

A detector unit, system or method as herein described wherein said communication system is a wireless communication system.

A detector unit, system or method as herein described wherein said radiation sensor cable is attached to a medical balloon, a rectal balloon, a vaginal balloon, a catheter, a urinary catheter, a cardiac catheter, a skin patch or a medical device for attachment to a body.

A detector unit, system or method as herein described wherein said connector is an SMA connector.

A detector unit, system or method as herein described further comprising a display screen on an exterior surface of said detector unit.

A detector unit, system or method as herein described further comprising a liquid crystal display screen on an exterior surface of said detector unit.

A detector unit, system or method as herein described wherein said shielded housing is shielded with lead, e.g., about 1-25 mm thick, 2-10 mm thick, 3-8 mm thick or about 5 or 6 mm thick.

A detector unit, system or method as herein described comprising a barcode reader on an exterior surface of said detector unit, and a barcode containing calibration information is provided with said radiation sensor cable. In preferred embodiments, patient information can also be encoded in a barcode, and place e.g., on the medical balloon or radiation sensor cable, or elsewhere.

A detector unit, system or method as herein described further comprising a processor operably coupled to said photodiode, said processor converting a signal from said radiation sensor cable to a radiation dosage.

A detector unit, system or method as herein described further comprising a processor operably coupled to an analog-to-digital converter (ADC) operably coupled to said photodiode, ADC converting said a signal from said radiation sensor cable to digital signal, and said processor converting a digital signal to a radiation dosage.

A detector unit, system or method as herein described further comprising a display for displaying said radiation dosage. Said display can be separate, or integral, e.g., on an outer surface of said detector unit, or both.

A detector unit, system or method as herein described wherein said radiation sensor cable is on an exterior surface of a rectal balloon, sized and shaped to immobilize a prostate when inflate adjacent a prostate. Preferably, the rectal balloon compresses the prostate, holding it in a steady position.

A detector unit, system or method as herein described said radiation sensor cable having a barcode on an exterior surface thereof or on packaging. If so, the method would include the step of reading the barcode before commencing treatment.

As used herein, the term “photodiode” is a single semiconductor device or an array of semiconductor devices, operating in non-Geiger or Geiger mode, whereby each photon generates a single count of electrons almost instantaneously and results in an electric current, often referred to as photo-electric current, that may be measured directly or converted into a voltage through an appropriately designed current-to-voltage electronic circuit. As used herein, the term “photodiode” specifically excludes a Charge-Coupled Device (CCD) which consists of a two-dimensional array of metal-oxide semiconductor capacitors where the photoelectrons generated within each capacitor are not read instantaneously, but instead charge individual capacitors and these charges are moved in the CCD circuit to allow charge to spill from one capacitor to the next.

As used herein, “directly abuts” means that the two surfaces are in direct contact or so nearly so as to not allow light loss between them. Typically, any gap between the two is so miniscule as to be difficult to visually detect. Specifically excluded is the use of epoxy between the two abutted surfaces, as well any free-space optics.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention, such as instructions for use, labels, and the like.

The following abbreviations are used herein:

| ABBREVIATION | TERM |
|---|---|
| ADC | analog-to-digital converter |
| CDU | Clinical Detector Unit |
| LCD | Liquid crystal display |
| PCB | printed-circuit-board |
| POF | Plastic optical fiber |
| PSD | Plastic Scintillating Detector |
| REST API | Representational State Transfer |
| | Application Program Interface |
| SMA | Sub Multi Assembly |

### BRIEF DESCRIPTION OF THE DRAWINGS

FIG. 1A-D. Monolithic photodiode detector. FIG. 1A presents a perspective view of an assembled monolithic photodiode detector. FIGS. 1B and 1C shows the system partially disassembled from two separate angles, so that the fiber optic connector receptacle and threaded connector can be more clearly seen. FIG. 1D shows a top view of the PCB and photodiode.

FIG. 2 Overall system architecture.

FIG. 3 Clinical Detector Unit.

FIG. 4 shows a 1×2 splitter splitting a POF into two.

FIG. 5 shows an alternative connection mechanism.

FIG. 6 shows an exemplary circuit diagram.

FIG. 7. Rectal balloon with radiation sensor cable in use in patient.

FIG. 8A. Details on one exemplary dual radiation sensor cable. FIGS. 8B and 8C show close up views of the two ends of the cable.

FIG. 9A is an exploded cross section of an embodiment of a skin patch sensor having cup with fiducial or visible markers on the edge of a wall thereof into which the oncologist places a conformal bolus.

FIG. 9B is an exploded cross section of one embodiment of a skin patch sensor showing its disc shape and printed central target and cross hairs on the upper surface thereof.

FIG. 10 shows a triple radiation sensor cable on a vaginal brachytherapy balloon.

FIG. 11 provides a picture of a balloon catheter equipped with the small diameter radiation sensor cable.

FIG. 12 provides an alternative embodiment, wherein the POF is directly abut to a two-color dichroic prism with the photodiode or MPPC surface directly abut to the reflection and transmission surfaces of the prism.

FIG. 13 provides an alternative embodiment, wherein the POF is directly abut to a three-color crossed dichroic prism with the photodiode or MPPC surface directly abut to the reflection and transmission surfaces of the prism.

### DETAILED DESCRIPTION

The disclosure provides a novel monolithic photodiode or MPPC based real time dosimeter. By “monolithic” herein we mean that the device is without free-space optics in the light optical train, so that the light is transmitted through a fiber optic cable; only exiting the POF via a fiber optic connector, e.g., the light does not travel through air, as in the prior art devices.

By “free space optics” what is meant is that the light travels through an empty space of at least 0.5 mm, usually more, rather than being directly abutted against the photodiode, MPPC, dichroic prism, or crossed dichroic prism.

The monolithic photodiodes and MPPC detectors described here can be used in many applications, including external beam radiation therapy (XRT), stereotactic radiosurgery/stereotactic radiotherapy (SRS/SRT), intensity modulated radiation therapy (IMRT), dynamical arc therapy, tomotherapy treatments, and any similar application where dosimetry is needed, as well as non-medical and scientific applications.

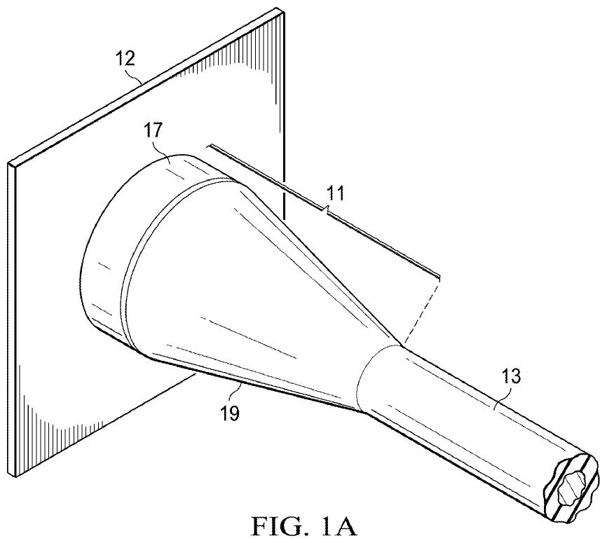

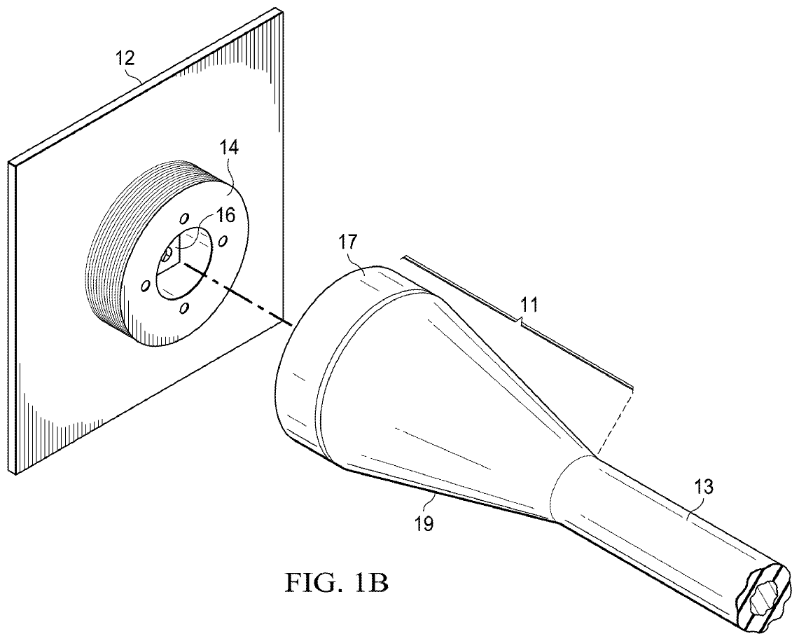

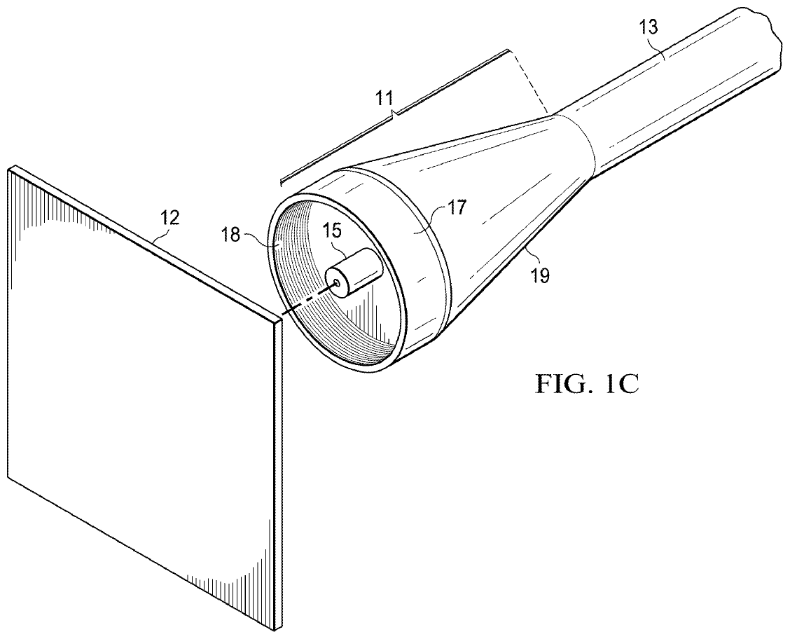

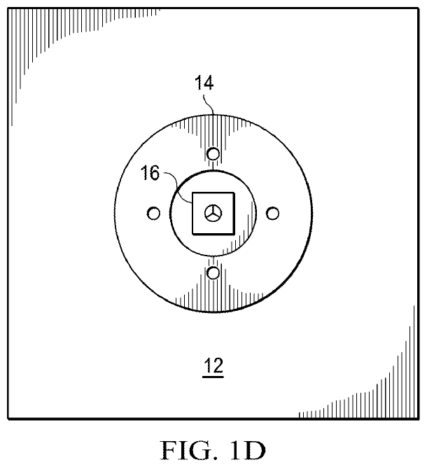

FIG. 1A-D shows a monolithic photodiode according to one embodiment of the present disclosure from several angles and the legend for these figures is in Table 1.

TABLE 1
LEGEND for FIG. 1

| | |
|---|---|
| 11 | SMA Fiber Optic Connector |
| 12 | Printed Circuit Board (PCB) |
| 13 | Fiber Jacket |
| 14 | Bulkhead SMA Receptacle |
| 15 | Fiber Optic Ferrule with encased fiber optic |
| 16 | RGB Photodiode mounted to PCB |
| 17 | Adapter |
| 18 | Threads on adapter |
| 19 | Strain relief rubber housing |

One embodiment of the present device has a SMA Fiber Optic Connector 11 that houses the end of a fiber optic cable from a Plastic Scintillating Detector (PSD) (not shown) and a printed circuit board (PCB) 12 housing a photodiode. This embodiment is shown fully assembled and connected in FIG. 1A and disconnected in FIG. 1B.

The SMA has a strain relief rubber housing 19 to protect the fiber optic cable from severe bending. The housing has two ends, one of which has an adapter 17 with means of attaching the SMA to a bulkhead receptacle 14 on a printed circuit board (PCB) 12 and the opposing end has the fiber optic cable entrance. Here, the adapter 17 is depicted as having a threaded attachment 18 in FIG. 1C for attachment to the bulkhead receptacle 14, but other attachments can be used such as snap fits, screws and the like. The purpose of the adapter 17 is to provide a precise mechanical tolerance and consistent aligned connection when attached to another fiber and/or component through a bulkhead receptacle.

The other end of the SMA Fiber Optic Connector 11 features an opening for receiving the fiber optic cable and a fiber jacket 13 that encloses the fiber optic cable and the edge of the SMA to protecting the fiber from stray light signals. The remainder of the sensor cable is omitted for simplicity.

The fiber optic cable enters the SMA and is mounted inside a ferrule 15 as shown in FIG. 1C. The ferrule is a small tube that the fiber slides into. Typically, epoxy is placed on the fiber optic side surfaces (not the end) and is slid into the ferrule. After the epoxy sets, the end of the fiber optic cable is polished down to the ferrule surface. The purpose of the ferrule is to provide a precise alignment of the light existing the optical fiber when screwed into a connector (in this case the bulkhead connector) on the PCB 12.

As mentioned above, the PCB 12 has a bulkhead receptacle 14 that is screwed or otherwise mounted onto the PCB 12. The RGB photodiode 16 rests inside the bulkhead connector and is aligned with the ferrule 15 on the SMA Fiber Optic Connector 11. Preferably, the POF and ferrule are positioned so that the clean end face of the POF abuts or nearly abuts the diode (e.g., having <0.5 mm separation, preferably <0.2 mm or <0.1 mm.) The photodiode 16 is affixed to a printed circuit board (PCB).

In this example, the RGB photodiode 16 is basically a series of three closely packed silicon detectors. The single chip includes three interference color filters, which only pass the given spectrum of light onto the respective photodiode. The light-sensing aperture is about 2-3 mm in diameter (encompassing all three detectors) and the fiber is 0.5-2 mm in diameter. When the SMA 11 is attached to the bulkhead receptacle 14, the surface of the fiber sits just above (<0.5 mm) the surface of the RGB. The light exits the fiber and spreads evenly across all three-color sensing elements with little to no loss.

Any photodiode can be used, provided it allows sufficient sensitivity and reduced noise at a good cost point, but RGB photodiodes are preferred. Exemplary RGB photodiodes are available, e.g., from Newport Corp. (Bozeman, Mont.), Precision Micro-Optics (Woburn Mass.), OSI Optoelectronics (Hawthorne, Calif.), among others. Suitable RGB PDs include the 818-xx-L, 818, 918D Series Low-Power Photodetectors, and legacy 918 Series detectors (Newport®). Hamamatsu photodiodes may be preferred as they make some of the best photodiodes currently available.

The HFD3033-002/XXX PIN Photodiode (Honeywell®) is designed for high speed use in fiber optic receivers. It has a large area detector, providing efficient response to 50-100 μm diameter fibers at wavelengths of 650 to 950 nanometers. Light is collected using a 600 micron micro lens mounted on the detector surface. The HFD3033-002/XXX is comprised of an HFD3033 PIN photodiode, which is mounted in a fiber optic connector that aligns the component's optical axis with the axis of the optical fiber.

The HFD3033-002/XXXs case is electrically isolated from the anode and cathode terminals to enhance the EMI/RFI shielding which increases the sensitivity and speed. The housing acts as a shield for the PIN photodiode component.

The bulkhead connector 14 is just a connector that allows for one type of fiber optic connector (here, the SMA) to be coupled to another. The back-side (or bottom) of the bulkhead receptacle 14 is open to allow light to pass directly onto the photodiode surface.

This fiber optic design provides a robust, monolithic design, which dramatically reduces the potential for damage during shipment, vibration, or transportation of the device.

Virtually any combination of connector and bulkhead mount could be used provided they are compatible, provide a sufficient level of mechanical alignment tolerance, have sufficient low sensitivity to vibration, and can be mounted to a PCB in a light tight enclosure. The SMA connector tends to be the most robust for the money and is still used widely, although it is slowly being replaced by ST, FC, SC connectors, all of which may be suitable. ST and FC connectors are more expensive, but otherwise provide similar functionality as the SMA connector. The SC connector is spring-loaded, and therefore may be less preferred as subject to damage during shipping. The FP3-SMA Fiber Connector Adapters accommodates optical fibers terminated with SMA connectors, but many other examples are available. Exemplary connectors and mounts combinations that can be used to create the PCB mounted PD include:

| | |
|---|---|
| SMA 905 Connector | HPLC-SMA/M Bulkhead receptacle |
| FC Connector | HPLC-NTT/FC-SM |
| ST Connector | HPLC-ATT/ST-SM-AL |

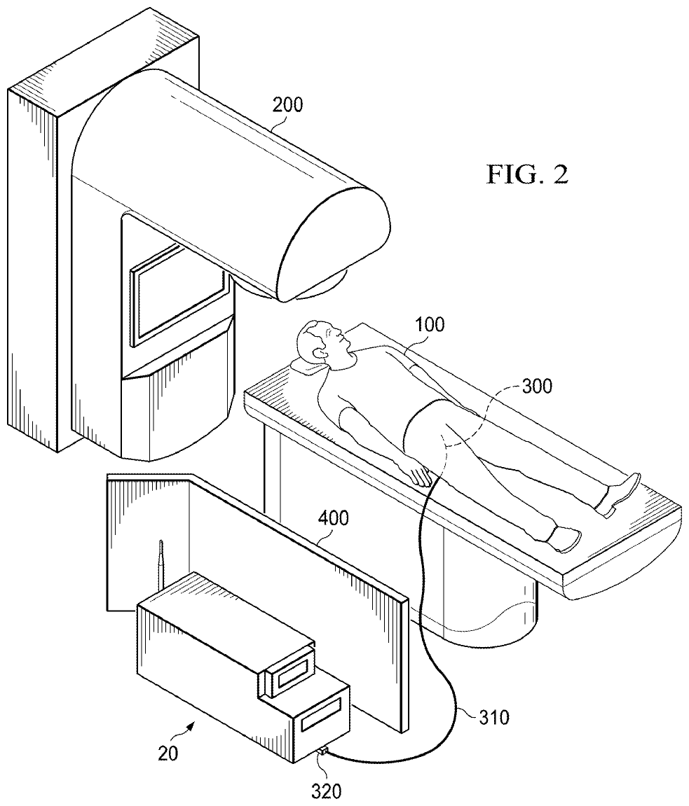

The complete system is shown in FIG. 2, wherein a patient 100 and XRT machine 200 are in a separate treatment bunker, needed to contain the radiation. The plastic scintillator dosimeter 300 (distal end not visible) is inserted into a body orifice of the patient 100 being treated, e.g., the rectum for XRT treatment of the prostate. The legend for FIGS. 2 and 3 is below in Table 2.

TABLE 2
LEGEND for FIGS. 2 and 3

| Item | Component | Component Description |
|---|---|---|
| 100 | Patient | |
| 200 | XRT machine | Typically a linear accelerator or |
| | | “linac” but other types of |
| | | machines are available |
| 300 | PSD | |
| 310 | PSD cable | |
| 320 | SMA connector | At proximal end of PSC, reversibly |
| | | connects to detector unit |
| 400 | Shielded wall | |
| 20 | Clinical Detector Unit | |
| | or CDU | |
| 21 | Housing | Encloses detector unit and protects |
| | | it from stray light |
| 22 | PCB | Printed circuit board |
| 23 | Cable connected to detector | |
| 24 | Inner lead shield housing | Shields sensitive electronics from |
| | | radiation |
| 26 | Micro-Processor | Process measurements and controls |
| | | CDU using ADC + proprietary soft- |
| | | ware and measurement process |
| 27 | PCB mounted Photo-Diode | Measures light from PSD sensor |
| | Sensor | |
| 28 | SMA | |
| 29 | Cable conversion adapter | |
| 30 | Assembled SMA and PCB | |
| | sensor | |
| 31 | LCD | Provides feedback on CDU status |
| 32 | Barcode Scanner | Scans PSD sensor barcodes |
| 33 | Power Supply/Battery | Re-Chargeable battery with AC/DC |
| | | Power Supply for powering the CDU |
| 34 | Ethernet Connection | Wired communication with CDU |
| 35 | Wi-Fi Antenna | Wireless communication with CDU |
| 36 | Standard 120 V socket | Charges battery and/or powers CDU |
| | | without battery |
| 37 | Local Network | Unique protected network facilitating |
| | | communication with CDU |

The clinical detector unit or “CDU” 20 is also in the treatment bunker, but shielded from the direct radiation (e.g., behind a shielded wall 400). This may not be needed, since the CDU 20 has its own internal shielding 24, but it is common in the art to place electronics at a sheltered position away from the XRT machine, and most bunkers will have such allocation of space equipped with power sources, lights, a desk and the like. Thus, it is anticipated that this location will be convenient in hospitals and clinics, even if not needed. The PSD cable 310 with SMA fiber optic connector 320 leads to the detector unit 20, here shown fully connected.

A close-up of one embodiment of the CDU 20 (a cut away view) is shown in FIG. 3. This embodiment showcases a design for single-use fiber applications. In particular, a PSD cable with a cheaper connector can be utilized for a single-use application. A cable conversion adapter 29 can be located inside the CDU 20 for converting the cheaper connector 23 to the e.g. SMA 28 or other connector in the CDU 20. This will keep cost down while maintaining the higher level of transmission for the SMA.

The Clinical Detector Unit or CDU 20 has a housing 21 that is opaque and encloses and protects the unit. A second interior housing is also provided, which serves to shield 24 the system from stray excitation. This shielding 24 is typically a lead lined enclosure, herein lead is 1-25 mm thick, or 2-8 or 3-10 or about 5 or 6 mm thick, or preferably about ¼-½ inch thick.

Inside the secondary housing or shielding 24 is a printed circuit board or PCB 22, onto which is mounted an RGB Photo-Diode Sensor 30, in the manner described in FIG. 1. The RGB Photo-Diode Sensor 30 includes the PCB mounted connector 25, the RGB sensor 27, and the SMA 28. As noted above, the SMA 28 has a fiber optical cable connecting it to a conversion adapter 29 that attaches to the single use PSD 23.

Micro-processor 26 processes measurements and controls the CDU using an on-board analog-to-digital converter or “ADC” and software and measurement process. Further, the microprocessor 26 will calculate the radiation dose and transmit the digital measurement of doses, preferably wirelessly, to any client device attached to the network 37. The PCB has a separate electric circuit from the micro-processor and ADC circuit, but the circuits will be connected through on-board wiring. The ADC is typically built into the micro-processor circuitry.

The on-board software calculates dose using an algorithm that converts light measured by the photo detectors into dose measured. The dose measurement process is insensitive to temperature effects of the PSD and Cherenkov radiation produced in the POF fiber. The software driver provides end-users with a standard REST API for interacting with the CDU. The REST API allows 3rd party software to both write and read from the CDU through a standard CATS/6 Ethernet cable, USB, (or other suitable cable) or via wireless transmission across the system network.

Applicants note that the POF fiber (as well as the PSD) inside the POF emits some light during radiation. However, a filter can be used to limit the light emitted from the POF and PSD through calibration of the detectors to overcome this issue. Essentially, when the sensors are calibrated: all cable dependent light emission is calibrated out.

The housing 21 preferably also has a LCD display 31 and barcode scanner 32 mounted on the front or other suitable surface of the housing 21. The LCD 31 provides feedback on CDU 20 status and shows the serial number of the PSD bar code scanned, and the currently measured dose in real-time.

An on-board barcode scanner 32 allows the end-user to scan calibrated sensor barcodes near the CDU 20. The therapist scans a barcode, which contains the individual calibration coefficients used by the software to calculate dose for each PSD. The system needs that information to convert the light measured into a meaningful dose measurement. The barcode can also contain patient related data or a separate barcode can be used for that, wherein the treatment coordinator provides these at the beginning of treatment and adds a second barcode to each cable (or balloon). Currently, the barcode scanner is located outside the treatment area, which is inconvenient since one must scan the barcode into software and then give the device to therapist to put into the patient. This adds flexibility so the therapist can simply scan the barcode right next to treatment table and then insert or apply the sensor cable to patient.

Power supply 33 or battery, e.g., rechargeable battery with AC/DC Power Supply for powering the CDU 20 is provided, but the device can also be plugged into a standard 120 Volt outlet 36. Power can be provided by any means, including Power over Ethernet (PoE) cable or by USB, if that is suitable for the location. Batteries may be preferred, and if so, a low battery detector can be included in the device with a low battery warning light on the surface thereof. In some embodiments, the battery may be outside the housing, or at least outside the interior shielded housing in order to facilitate changing the battery.

Ethernet Connection or USB connection 34 is provided for wired communication with the CDU 20, but Wi-Fi Antenna 35 provides wireless communication with CDU. It may be preferred to provide both options, as different facilities will have different layouts. Providing both an Ethernet or USB connector and wireless communication system allows the user to customize usage according to needs. Alternatively, a single option can be provided to reduce costs and the user selects which model to purchase.

The antenna 35 screws onto the outer frame of the CDU 20 through an RF connector. The RF connector has a wire (not shown) that will be connected to the ADC circuit. A local area network, or LAN, 37 provides a unique protected network facilitating communication with CDU. Thus, the CDU 20 can communicate with any computer on the LAN, and does not need a direct cable connector, which is subject to damage and extremely inconvenient to install given that radiation treatment is restricted to shielded bunkers in order to protect the technicians from radiation.

The system as shown in FIG. 3, provides radiation oncologists and physicists with real-time, in-vivo, multi-point radiation-dose information obtained from e.g., Radiatrac® Plastic Scintillating Detectors (PSD) sensors to monitor dose photon based radiation therapy for cancer treatment. This information allows the physician to monitor the dose during patient treatments, compare the actual dose relative to the planned dose, and provides graphs and dose information of the cumulative and instantaneous dose for the current treatment. Dose data is time-resolved at up to ˜500 samples/s (500 Hz), whereas the prior art CCD based system require about 20 seconds per datapoint. Thus, the system is some 10,000 times faster than the prior art systems. In addition, the system allows the end-user to save the time-resolved dose information to disk for further analysis.

Though the above embodiments are described using an SMA Fiber Optic Connector, other types of connectors can be used including ST, FC, SC, SCRJ and SMI connectors.

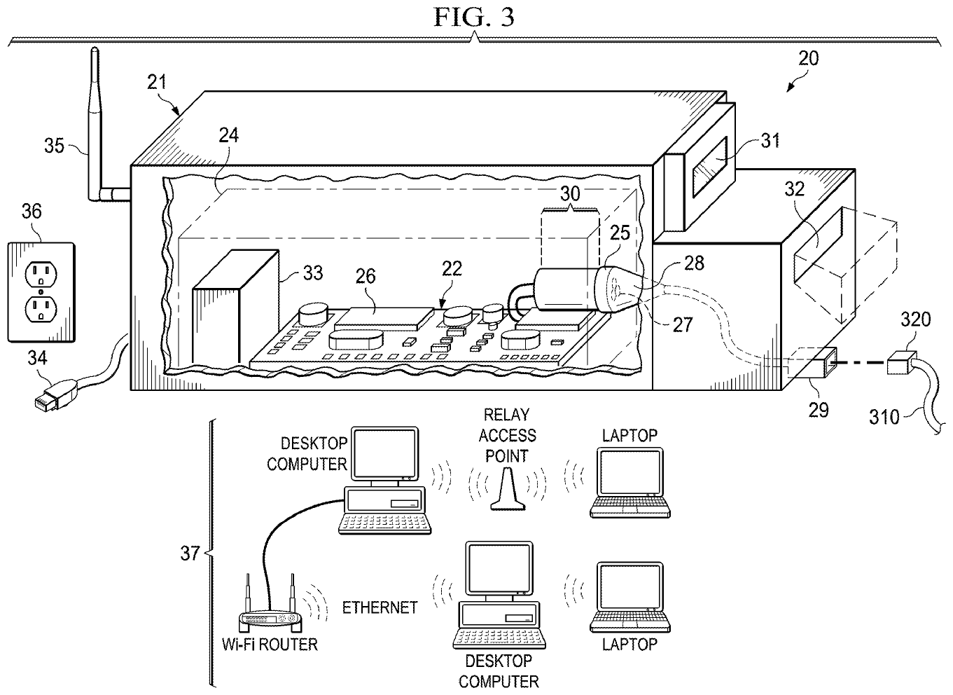

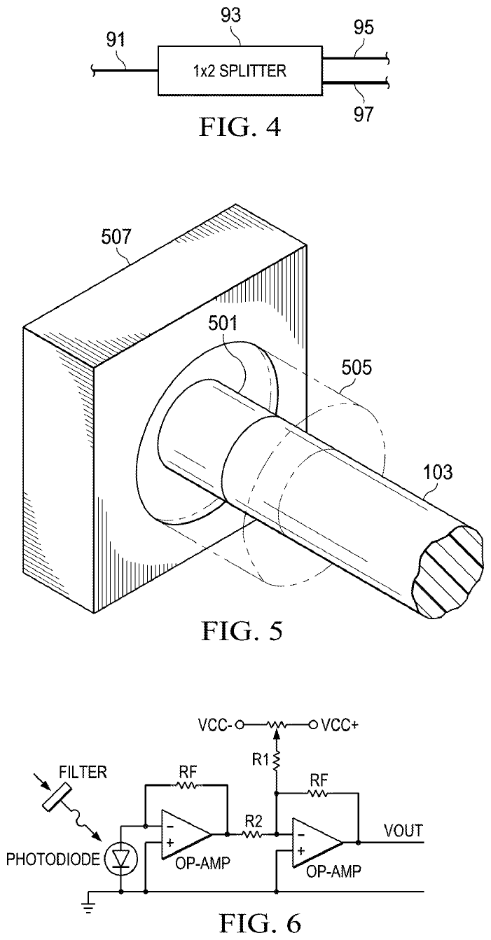

In other embodiments, the connecting end of the fiber optic cable may be split through a low-loss fiber optic multiplexer (e.g., FIG. 4) and guided onto a series of photodiodes using the same housing type listed in [0060] using two or more ferrules. FIG. 4 shows fiber optic cable 91 entering a 1×2 splitter 93, which splits the signal into two fiber optic cables 95, 97.

In yet other embodiments, the connecting end of the fiber optic cable may be split through a low-loss fiber optic multiplexer and directly mounted to a photodiode assembly. The SMA would thus be omitted, and the bare fibers epoxied onto the photodiode surface, as in FIG. 5. The photodiode 501 is mounted to the PCB 507. The bare POF 103 is epoxy mounted to a tube or housing 505, e.g., an aluminum tube, for durability. The aluminum tube is mounted to the PCB using epoxy or threads. The fiber optic cable surface abuts or nearly abuts the photodiode surface.

The device's sensor cable is connected to the Clinical Detector Unit (CDU) consisting of photo-diodes and state-of-the-art trans-impedance low-noise amplifier electrical circuits built onto a printed-circuit-board (PCB), per e.g., FIG. 6, which shows an exemplary circuit only.

The photo-diodes are equipped with infrared (IR) blocking filters as well as color filters; and contained within the RGB housing. The electronic signals from the photo-diodes are buffered, conditioned, and adjusted for temperature changes to provide increased stability. The signals are connected to a dedicated microprocessor equipped with an on-board analog-to-digital converter (ADC) and software drivers.

The photodiode circuit (Printed Circuit Board) has output pins to carry an analog voltage signal (which is directly proportional to the light incidence on the photodiode) to the microprocessor and ADC circuit (usually another PCB). These pins may be either 1) connected to the microprocessor and ADC circuit via electrical wires or 2) permanently attached to the photodiode PCB through soldering. However, these details can vary and the actual setup may change based on space and hardware considerations, as is known in the art.

The system improves upon existing dosimeter monitoring system in several ways. The monolithic all-fiber based architecture eliminates any susceptibility to misalignment or shifting of free-space optics during shipment of the CDU from manufacturer to customer. This increases the stability and robustness of the dosimeter system.

The system preferably also eliminates the delivery fiber optic cable (robust cable), which is susceptible to damage during installation and during normal operation. This cable is typically needed do carry the light signal from the PSD out of the radiation treatment vault and into the CDU. It is difficult to install in existing facilities, is subject to damage and allows additional noise input and light signal attenuation.

Together with the on-board electronic adjustments, each CDU may also be normalized and cross-calibrated using a calibrated light source before shipment to the customer. This eliminates the time-consuming task of commissioning each CDU using “constancy” sensors together with a radiation source. Due to this shortcoming, the current OARtrac® system may only be commissioned by a designated calibration facility.

This system, in contrast, allows for on-site re-calibration using a calibrated light source. In addition, since the normalization procedure is made using hardware adjustments, the CDU is completely de-coupled from the computer or device interfacing with it; allowing for any CDU to be used with any computer or device. With the current OARtrac® system, the normalization coefficients are stored locally on the OARtrac computer, thereby creating a dependent relationship between each CDU and computer.

Replacing the charge-coupled-device (CCD) with a photodiode or MPPC also offers several technological improvements including a significant reduction in the dose measurement time from 20 seconds to 0.002 second, a simplified measurement and calibration process which eliminates the need to establish spatial regions of interest (ROIs), increased signal-to-noise ratio by directly coupling fiber output onto photodiode or MPPC, and a substantial reduction in both the overall size and manufacturing cost of the system.

With the current OARtrac® system, an overall thermal correction factor must be applied during the measurement process to correct for any temperature change in the PSD relative to room temperature. Because it is cost-prohibitive to apply a thermal correction to each PSD, an “average” thermal correction factor is applied which reduces overall dose accuracy of the system. The present design virtually eliminates the need to correct for the inherent thermal dependence of the PSD, thereby increasing overall dose accuracy and reducing system complexity.

The present OARtrac® system, which, if installed permanently, requires a fiber optic delivery cable to be routed through the treatment vault or run under the treatment door is susceptible to changes in system response due to tight bends of the fiber and/or damage caused by stepping on the cable or improper handling.

The present invention however, enables dose to be measured wirelessly or through an Ethernet or USB cable. The former, coupled with an on-board battery powered CDU, creates a cable-free, modular, and portable system, which is easily transported in and around the treatment vault or radiation environment. Even in cases where an Ethernet cable or USB must be used, the installment of the system only requires routing an electrical cable through the radiation vault. Stepping on the Ethernet cable or USB or tight bending is not expected to cause any change in dose measured since a digital signal is transmitted along the electrical wires.

Table 3 compares the technology and summarizes technological improvements and advancements of the present invention over the current version of OARtrac®.

TABLE 3
System Comparison between prior OarTrac ® CCD
based system and improvements of the invention

| Item | OarTrac ® | Present Invention |
|---|---|---|
| Measurement Architecture | Free-Space Optics | Monolithic all-fiber based |
| | and CCD | and Photodiode or MPPC |
| Susceptible to system changes due to | YES | NO, fiber based system |
| misalignment and shifting during | | allows direct coupling onto |
| shipment | | Photodiode or MPPC surface |
| Requires designated facility with | YES | NO, system may be |
| radiation for system commissioning | | commissioned on-site using |
| and normalization | | calibrated light source |
| Delivery Fiber Optic Cable | YES | NO |
| Requires designated computer for | YES | NO, any computer can be |
| each CDU | | used with any CDU |
| Susceptible to changes in system | YES | NO |
| response due to fiber optic cable | | |
| damage or changes | | |
| Dose Measurement Time | 20 (s)/measurement | <1 (s)/measurement |
| Requires designation of spatial regions | YES | NO |
| of interest (ROIs) | | |
| Size Footprint | ~1′ × 6″ × 6″ | ~4″ × 4″ × 2″ |
| Weight | 10 lbs | <0.5 lbs |
| | (No Shielding) | (No Shielding) |
| Requires thermal correction for Dose | YES | NO |
| due to PSD temperature dependence | | |
| Wireless dose measurement | NO | YES |

The present invention is exemplified with respect to the recently approved OarTrac® radiation sensor cable. However, this is exemplary only, and the invention can be broadly applied to any radiation sensor cable. Further, the invention may have applications outside dosimeter use, such as measuring radiation in sterilization units, measuring radiation in a scientific or research context, and the like. Additionally, although designed for external beam radiation therapy (XRT), such devices may also have uses in brachytherapy and other radiation based treatments.

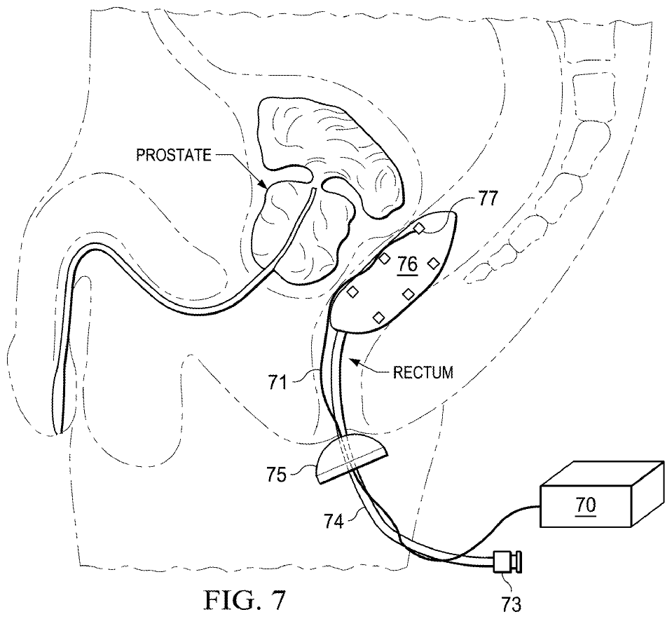

FIG. 7 shows one exemplary use, wherein a rectal balloon 76 is inserted into the rectum of a male patient and positioned adjacent the prostate. Preferably, the balloon is shaped and sized so as to compress the prostate thus immobilizing it for XRT treatment. Next, the balloon is inflated, via stop cock or luer connector 73 on inflation lumen 74. Fiducial markers 77 may be visualized, e.g., with MRI or other medical imager, to ensure the balloon is reliably positioned. Once reliably positioned, stopper 75 is locked in position, preventing the balloon 76 from being drawn inward.

The radiation sensor cable 71 lies on the prostate side of the balloon, either on a surface thereof or in a channel on the surface (not shown). The cable travels down the lumen 74, and plugs into the detector unit 70, described above.

The patient is then treated, typically with external beam radiation therapy, and the real time dosage being delivered can be monitored. Radiation can be stopped when it has reached the desired level for a particular treatment session.

If desired, a rectal balloon having a gas release lumen allowing gas to bypass the balloon can be used, as described in more detail in US20120123185 and US20100145379. If so, the method includes the added step of slow insertion and carefully releasing all gas before inflation, inflation, and then continuing to release gas during treatment. This method has been clinically proven to reduce prostate motion, allowing further reduction in treatment margins.

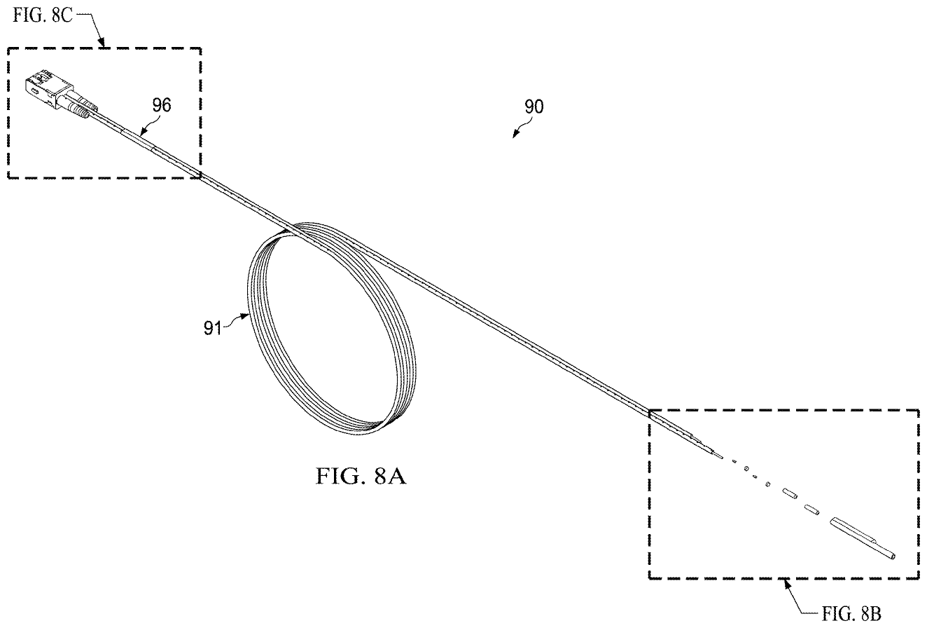

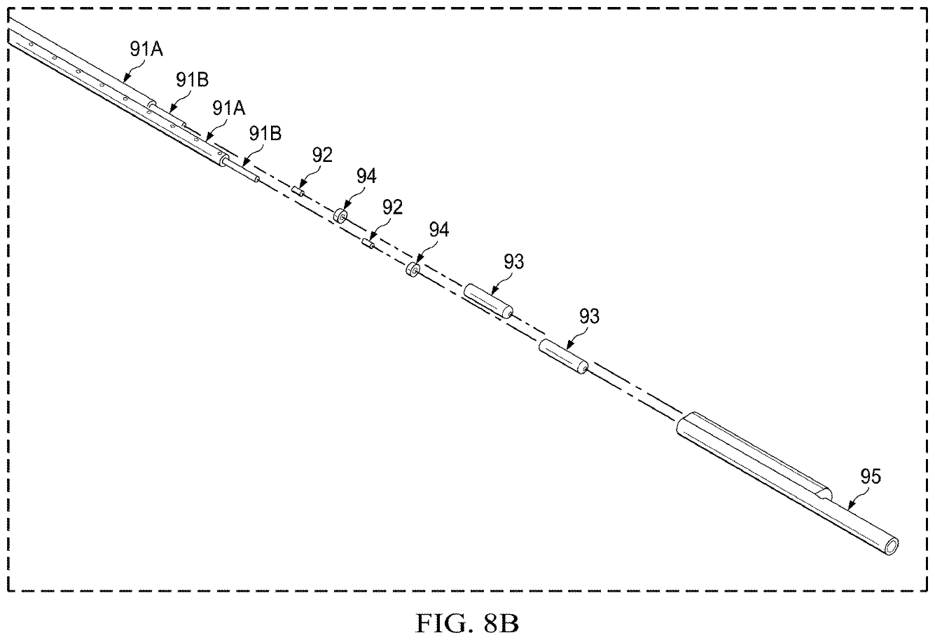

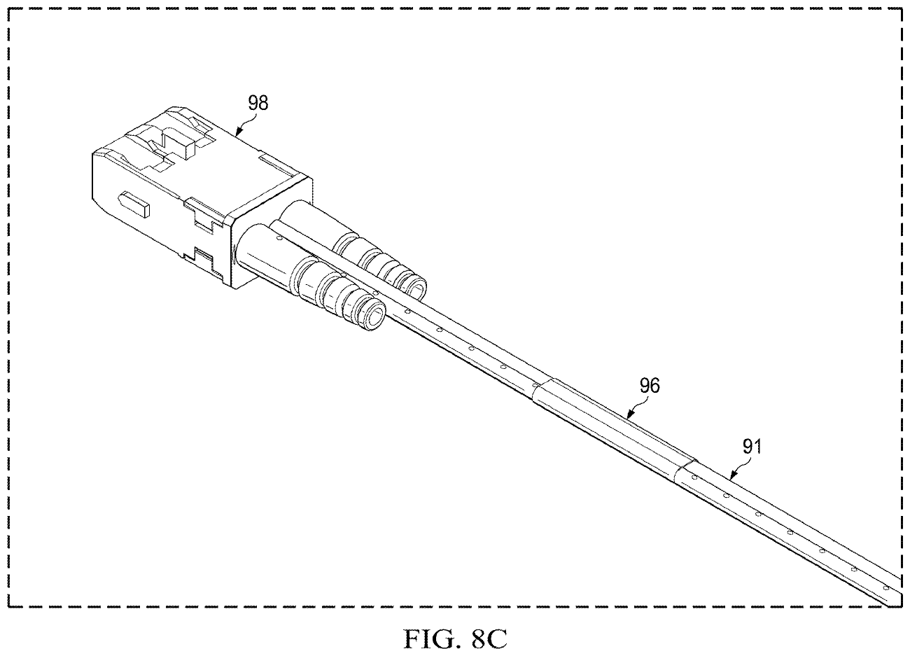

FIG. 8A shows one embodiment of an assembled radiation sensor 90, while FIG. 8B shows the exploded sensor components and FIG. 8C shows the connector 98 at the proximal end of the cable. The device and cap is described in more detail US20120281945.

In FIG. 8B a duplex scintillation detector cable 90 has a first and second optical fibers 91, but the same principals can be used for varying number of sensors. The jacket or covering 91A has been stripped or removed from the portion of the first optical fiber 91 adjacent to the distal ends of each fiber, leaving a portion of each optical fiber 91B exposed. First and second scintillating fibers 92 are shown, along with drop of adhesive 94 and fiber cap 93. The length of scintillating fibers 92 can be varied, according to needed sensitivity and size of area to be assessed, but typically 1-10 mm or 2-3 mm of length will suffice. Of course, a single sensor cable could be used instead of a dual sensor cable, and more detectors can be added, subject to size limitations. For example, a urinary catheter sensor cable has a limited size, since the catheter is 2-4 mm, and the cable must fit within this limit.

The scintillating fibers 92 fit into the fiber caps 93, followed by the naked optic fibers 91B, and a drop of epoxy 94 on the sides (not ends). Heat shrink tubing 95 covers the components. At the far end, an adaptor 98 is found, in this case a dual jack adaptor. Label 96 is also shown, but may be placed anywhere on the cable or even on packaging and is not considered material. There is no adhesive 94 on the abutted ends or faces of the respective scintillating fibers 92 and optical fibers 91, thus signal are reliability are both optimized.

The duplex optical fiber 91 may be a Super Eska 1 mm duplex plastic optical fiber SH4002 available from Mitsubishi Rayon Co., Ltd. of Tokyo, Japan, although other duplex optical fibers are also contemplated. Although duplex optical fibers 91 are shown, it is also contemplated that a single optical fiber may be used or additional fibers can be added.

The scintillating fibers 92 may be a BCF-60 scintillating fiber peak emission 530 NM available from SAINT-GOBAIN CERAMICS & PLASTICS™, Inc. of Hiram, Ohio, although other scintillating fibers are also contemplated.

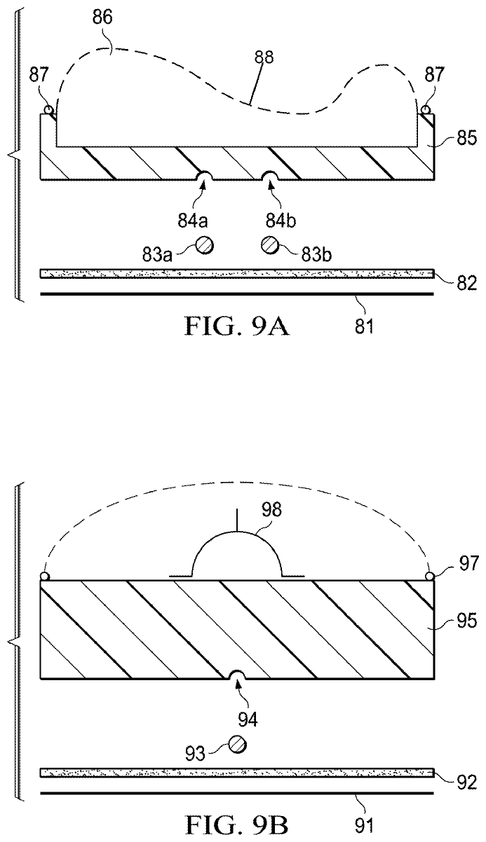

An embodiment of a dosimeter skin patch sensor is shown in exploded cross sectional view in FIG. 9A, wherein the patch has two radiation sensor cables, as herein described. A base 85 has grooves 84a and 84b into which fits the proximal tip of the sensor 83a and 83b. An adhesive layer 82 covers the sensor 83a, b, grooves 84a, b, to the extent needed, and bottom surface of the base 85. A separate bolus 86 material can be fitted into the cup provided in the upper surface by wall 88 by the oncologist or technician, or the bolus material can be provided with the patch as sold. A peel-off protective layer 81 protects the adhesive until use, at which time it is removed, and the patch attached to the patient in the target treatment area. In this example, fiducial and/or visible markers 87 are provided on the tops of the walls, such that they are clearly visible for alignment purposes.

By “bolus” herein what is meant is a water equivalent material that assists in evening the dose provided to the body and/or controlling the depth of the dosage. Preferred bolus materials are moldable, such that they can be shaped by the user.

Bolus materials can be any known or to be developed. Available bolus materials include Aquaplast RT Thermoplastic, which is 2-oxepanone, polymer with 1,4-butanediol (synonyms: Caprolactone, 1,4-butanediol polymer epsilon-Caprolactone, or 1,4-butanediol polyester) (WFR/Aquaplast Corp., Wyckoff, N.J., USA). This material has been shown as an effective bolus material, with thicknesses of 0.5 cm or 1 cm, Aquaplast RT Thermoplastic shows less than 2% of difference in comparison with polystyrene or superflab boluses, two commonly used bolus materials, when irradiated with 6 to 12 MV photon using a 10 cm×10 cm field size.

Other bolus materials include Polyflex, a hydrocolloid from DenstsPly®, or Jeltrate® Plus, also from DentsPly®. Other materials investigated for bolus use include solid water, paraffin, superflab, wet gauze, wet sheets, Play-Doh™, and gauze embedded with petroleum jelly.

FIG. 9B shows yet another variation where the entire base 95 is made of water equivalent material that is somewhat flexible such that the disc shaped patch can be fitted to any part of the skin. The upper surface of the base 95 has a target area and cross hairs printed thereon for visual alignment purposes, and fiducial markers 97 serve to allow alignment by other means. Dosimeter sensor cable 93, adhesive 92, cover 91 are also seen.

If desired, base material can be thermoplastic, such that it can be molded when heat is applied, thus forming a permanent shape when cooled. Such devices can be used throughout treatment on the same patient, ensuring reproducibility of the bolus shape between treatments. As another example, a microwave absorbing additive can be added to the matrix of the polymer and the patch microwave heated for shaping. These methods assume that the sensor and groove are heat and/or microwave resistant, such that the sensor fitting remains without air pockets and secure. If not, the base can have an upper layer which is shaped, cooled and attached to the base, e.g., via adhesives or snap fitting into a cup, or pressed onto tiny hooks while still warm, and the like. For example, a base can be provided with adhesive on both upper and lower surfaces, the upper adhesive used to attached the conformal bolus.

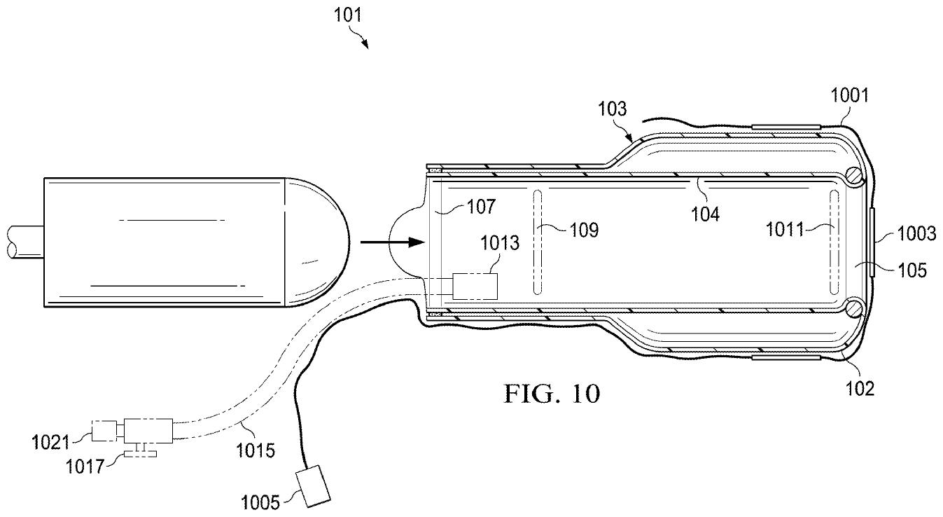

FIG. 10 shows a cross sectional view of a universal brachytherapy balloon 101, with toroidal balloon 103 having a cylindrical inner side 104 and a larger cyclindrical outer side 102, wherein the larger portion is at the distal end in a vaginal balloon, but whose shape can be modified according to the intended use. Welds 107, 109, 1011 are shown, as well as retention ring 105. Also shown are optional pull tabs 1019 at the proximal ends that assist the practitioner in inserting the applicator. The toroidal balloon 101 is fitted to a lumen 1015, in this case via a low profile doghouse 1013, and the lumen fitted with valve means 1017 for containing the air pressure on inflation, and optional syringe connecting means 1021. This balloon and its manufacture are described in additional detail in US20130085315, incorporated by reference herein in its entirety for all purposes.

The placement and spacing of the sensors can be customized for specific applications, but in a rectal balloon sits on or under the surface adjacent the prostate (anterior side). In a vaginal balloon, the sensor may fit at the distal end in the center where the cervix is being treated, and/or can be on the distal sides. FIG. 10 in fact shows a triple sensor cable 1001, each having a scintillating fiber tip 1003 and having an adaptor 1005 for connecting to the detector at the proximal end. A channel (not shown) can be provided to house the cable.

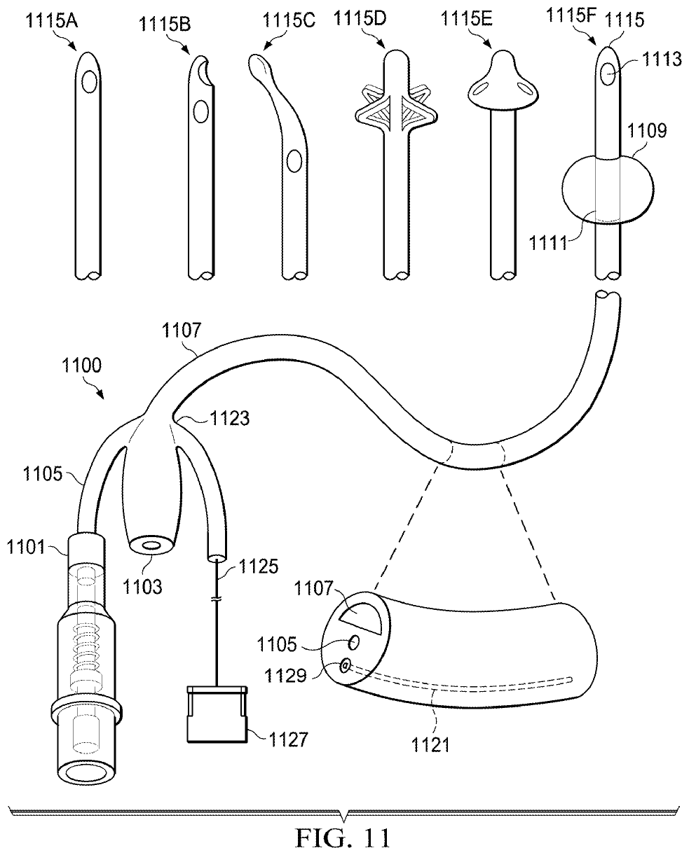

FIG. 11 provides a picture of a urinary balloon catheter equipped with the small diameter radiation sensor cable invented herein. The catheter 1111, has a trifurcated proximal end, a balloon 1109 near the distal tip, a capped tip 1115 with a port adjacent thereto, but proximal to the tip 1115. Fiducial marker 1115F can be placed at the tip and used for imaging the catheter 1111. The balloon 1109 anchors the catheter in the bladder, and the port 1113 allows fluid exit of entrance.

One end comprises an inflation valve 1101, usually a one way check valve often having luer lock fittings for connection to a syringe. A second end has a urine bag connector 1103, and the third end has a sealed outlet 1123 for the end 1125 of the sensor cable 1121, which terminates in an adaptor 1127.

In the embodiment of FIG. 11, a separate lumen 1123 is provided for the cable in which case the outlet need not be air-tight. However, where the air passageway 1105 and cable 1121 share the same passageway, the outlet 1129 should be air tight, such that air cannot escape the balloon, yet in preferred embodiments will still allow some adjustment of sensor position inside the catheter. A elastomeric seal will provide the necessary seal for outlet 1123, yet allow adjustment of cable position therein.

In other embodiments, the cable is small enough to fit under a strip of adhesive tape on the outside of the catheter (not shown), but this may not be preferred as a less robust and less smooth catheter may result, and a smooth exterior is preferred for urinary catheters. However, with the right selection of materials, this method may be acceptable.

In the small cross section of the catheter, can be seen the inflation lumen 1105 and the urine lumen 1107 and cable lumen 1123. The sensor cable 1121 can either be positioned in the inflation lumen 1105, or an additional lumen 1123 can be provided, as shown here.

As yet another alternative, the balloon and inflation valve can be omitted and the second lumen dedicated to sensor use.

Any tip can be used with the catheter of the invention, including 1115A—Simple urethral catheter; 1115B—Open-ended (whistle-tip) catheter; 1115C—Coude Catheter (Tiemann); Catheter 1115D—Wing-tip (Malecot) catheter; or 1115E—Mushroom (de Pezzer) catheter. However, the Tiemann tip may be preferred because it is designed to accommodated the enlarged prostate that occurs with benign and metastatic prostate cancers. Other variations of a catheter design are described in 62/063,196, URINARY RADIATION SENSOR CATHETER, Oct. 13, 2014, incorporated by reference herein in its entirety for all purposes.

In addition to being attached to e.g., a skin patch or medical balloon or medical catheter, the radiation sensor cable can be attached to other devices used to securely position the cable for use in radiation treatment, such as a limb splint for a limb cancer, a bite block for oral cancer, headgear for a brain tumor, and the like.

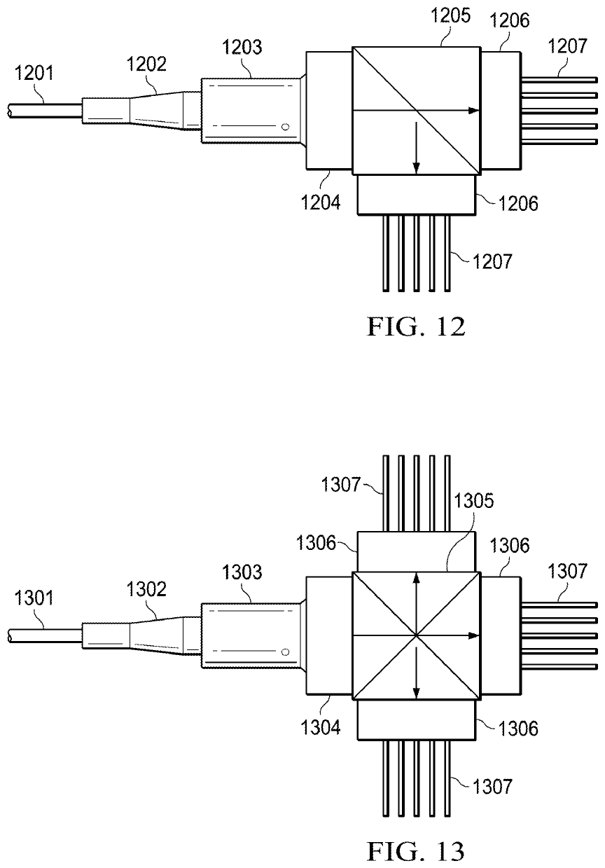

FIG. 12 and FIG. 13 show a variations of the embodiment of the invention, which includes using separate photodiodes or MPPC detectors in conjunction with a two-color dichroic prism or three-color crossed dichroic prism. Here, the POF is placed into a cylindrical aluminum housing using epoxy. The housing is placed into a flange whereby the end face of the POF is abut or slightly above (<0.5 mm) to the entrance face of the two-color dichroic or three-color crossed dichroic prism.

A photodiode or MPPC detector is directly abutted to the other transmitting and reflecting faces of the prism. The electrical leads then carry the electrical signal to the PCB. The entire outer assembly is impervious to light and the detector still uses a monolithic architecture.

In more detail, FIG. 12 shows 1201 POF connected to 1202 strain relief, used to protect the fiber from severe bending, connected to 1203 aluminum cylindrical housing and 1204 aluminum flange. The light from the POF 1201 travels through 1205 dichroic prism which splits the light into two separate optical light beams, each consisting of two spectral color regions, which are then guided onto and from thence to the 1206 PHOTODIODE/MPPC surface. Numeral 1207 represents the leads to the PCB (not shown).

FIG. 13 is similar with 1301 POF connected to 1302 strain relief, used to protect the fiber from severe bending, connected to 1303 aluminum cylindrical housing and 1304 aluminum flange. The light from the POF 1301 travels through 1305 a crossed dichroic prism, which splits the light into three separate optical light beams, each consisting of three spectral color regions, which are then guided onto and from thence to the 1306 PHOTODIODE/MPPC surface. Leads 1307 are connected to the PCB (not shown).

U.S. Pat. No. 8,953,912 SMALL DIAMETER RADIATION SENSOR CABLE

US20140051968 RECTAL BALLOON WITH SENSOR CABLE

US20120259197 RECTAL BALLOON WITH RADIATION SENSOR AND/OR MARKERS

US20120123185 US20100145379 RECTAL BALLOON APPARATUS WITH PRESSURE RELIEVING LUMEN AND SENSORS

US20140243580 ENDORECTAL BALLOON WITH GAS RELEASE LUMEN

US20120078177 US20100179582 Minimally invasive rectal balloon apparatus

U.S. Pat. No. 8,735,828 US20120068075, US20140221724 Real-time in vivo radiation dosimetry using scintillation detectors

U.S. Pat. Nos. 7,663,123 8,119,979, 8,344,335, US20090014665, US20100096540, US20120037808 Fiber optic dosimeter

US20130085315 UNIVERSAL BALLOON FOR BRACHYTHERAPY PPLICATOR

62/063,196, URINARY RADIATION SENSOR CATHETER, Oct. 13, 2014.

Klien, D. M., et al., “Measuring output factors of small fields formed by collimator jaws and multileaf collimator using plastic scintillation detectors” Med Phys., 2010, 37(10): 5541-5549.

Beddar A. S, et al., “A miniature “scintillator-fiberoptic-PMT” detector system for the dosimetry of small fields in stereotactic radiosurgery,” Nuclear Science, 2001, vol. 48, No. 3, pp. 924,928.

Klein D, et al., “In-phantom dose verification of prostate IMRT and VMAT deliveries using plastic scintillation detectors”, Radiat Meas., 2012, 47(10):921-929.

Beddar A. S. et al, “Water-equivalent plastic scintillation detectors for high-energy beam dosimetry: I. Physical characteristics and theoretical considerations”, Phys. Med. Biol., 1992, 37: 1883-1900.

Fluhs D., et al., “Direct reading measurement of absorbed dose with plastic scintillators—the general concept and applications to ophthalmic plaque dosimetry”, Med Phys. 23(3):427-304, March 1996.

Beddar A. S., et al., “Plastic scintillation dosimetry: optimization of light collection efficiency”, Phys Med Biol. 48 (9):1141-52, 2003.

Klawikowski S. J., et al., Phys Med Biol. 2014 May 7; 59(9):N27-36. Preliminary evaluation of the dosimetric accuracy of the in vivo plastic scintillation detector OARtrac system for prostate cancer treatments.

Alcon E. P., “EPR study of radiation stability of organic plastic scintillator for cardiovascular brachytherapy Sr90-Y90 beta dosimetry”, Appl Radiat Isot. 62(2):301-6, 2005.

1. A dosimetry detector system, comprising
a housing comprising a light sensor and a processor;
a radiation sensor cable comprising a scintillating fiber;
wherein the light sensor is configured to measure light emitted by the scintillating fiber;
wherein the light sensor is configured to generate an electrical signal in response to the measured light; and
wherein the processor is configured to convert the electrical signal into a calculated radiation dose measurement in less than 1 second.

2. The dosimetry detector system of claim 1, wherein the processor is configured calculate the radiation dose measurement in at least 0.002 seconds.

3. The dosimetry detector system of claim 1, further comprising:
wherein the calculated radiation dose measurement is time-resolved at a rate of up to at least 500 measurements per second.

4. The dosimetry detector system of claim 1, wherein the light sensor is a multi-pixel photon counter, a photodiode or a photomultiplier tube.

5. The dosimetry detector system of claim 1, further comprising a display unit.

6. The dosimetry detector system of claim 5, wherein the display unit is configured to display the calculated radiation dose measurement to a user in real-time.

7. The dosimetry detector system of claim 1, wherein the radiation sensor cable is configured to be operably connected to the housing.

8. The dosimetry detector system of claim 1, wherein the housing further comprises a power supply.

9. The dosimetry detector system of claim 1, wherein the radiation sensor cable is configured to be calibrated.

10. The dosimetry detector system of claim 1, wherein the housing further comprises a lead shielding.

11. The dosimetry detector system of claim 1, wherein the electrical signal may comprise electrons or photo-electrons.

12. The dosimetry detector system of claim 8, wherein the power supply is a battery.

13. The dosimetry detector system of claim 1, wherein the scintillating fiber is configured to emit light in response to radiation exposure.

14. The dosimetry detector system of claim 1, further comprising a filter.

15. The dosimetry detector system of claim 14, wherein the filter comprises a mirror configured to split the measured light into at least two-color regions.

16. The dosimetry detector system of claim 1, wherein the calculated radiation dose measurement is compared with an actual radiation dose delivered to a patient.

17. The dosimetry detector system of claim 1, wherein the calculated radiation dose measurement is compared with a planned radiation dose to be delivered to the patient.

18. The dosimetry detector system of claim 14, wherein the filter comprises at least two mirrors configured to split the measured light into at least three-color regions.

19. A dosimetry detector device, comprising
a housing comprising a light sensor, a trans-impedance amplifier, at least one filter, and a processor;
wherein the light sensor is configured to measure a signal;
wherein the trans-impedance amplifier and the at least one filter are configured to alter the signal measured by the light sensor; and
wherein the processor is configured to convert the altered signal into a calculated radiation dose measurement in less than 1 second.

20. A dosimetry device, comprising
a housing comprising a light sensor and a processor,
wherein the light sensor is configured to measure light energy emitted from a scintillator;
wherein the light sensor is configured to alter the light energy emitted from the scintillator into an altered signal;
wherein the processor is configured to convert the altered signal into a calculated radiation dose measurement in less than 1 second.
